SEQUENCE LISTING (1 sequence)

SEQ ID NO: 1 - protein, 39 aa, Heloderma suspectum
HGEGTFTSDLSKQMEEEAVRLFIEWLKNGGPSSGAPPPS

(19) United States
(12) Patent Application Publication
(10) Pub. No.: US 2020/0093736 A1
(43) Pub. Date: Mar. 26, 2020

(54) EXTRUDED DEPOT FORM FOR CONTINUING ACTIVE SUBSTANCE RELEASE

(71) Applicant: AMW GMBH, Warngau (DE)

(72) Inventor: Alexandra PARTENHAUSER, Kolbermoor (DE)

(73) Assignee: AMW GMBH, Warngau (DE)

(21) Appl. No.: 16/495,756

(22) PCT Filed: Mar. 22, 2018
(86) PCT No.: PCT/EP2018/057378

§ 371 Date: Sep. 19, 2019

(30) Foreign Application Priority Data
Mar. 22, 2017 (DE) 10 2017 106 216.5

(51) Int. Cl. A61K 9/00; A61K 9/16

(52) U.S. Cl. CPC A61K 9/0024; A61K 9/1617

(57) ABSTRACT

The invention relates to an extruded depot form for a continuing active substance release, comprising at least one active substance and at least two compounds of the class of substances which can be broken down by lipases. The at least two compounds comprise a low-melting compound and a high-melting compound, and the ratio of the low-melting compound to the high-melting compound ranges from 1:9 to 9:1. The depot form also optionally comprises at least one auxiliary agent for modulating the active substance release, wherein b), or optionally b) and c), constitute at least 60 wt. % of the dry weight of the depot form.

The present application relates to an extruded depot form, comprising at least one active substance and a combination of at least two compounds of the class of substances which can be broken down by lipases, wherein the at least two compounds comprise a low-melting compound and a high-melting compound. The present invention also relates to a method for producing the extruded depot form and to the use of the extruded depot form.

Subcutaneous administration forms generally comprise liquid or solid formulations which can be administered into the subcutaneous tissue by injection or surgical intervention. Solid formulations are usually administered in the form of cylindrical polymer rods with active substance embedded therein. Administration forms which deliver active substances over a period of time of a number of days up to, for example, 24 months are also referred to as depot dosage forms.

Active substances delivered by depot dosage forms are not subject to what is known as the first-pass effect, that is to say they do not pass through the digestive tract or the liver and in addition can ensure a continuous delivery of active substance over a prolonged period of time. In this way, strong fluctuations of the active substance concentration and therefore associated side effects, which often occur for example in the case of intravenous administration forms, are avoided. The controlled, sustained active substance release from depot dosage forms (also referred to hereinafter as “depot forms”) may additionally extend the application interval. Furthermore, depot dosage forms which are biodegradable no longer have to be removed after the intended application time. These properties mean that subcutaneous depot forms can be considered as user-friendly medicinal products.

The active substance release rate and duration from depot forms can be influenced by additives contained in the formulation, wherein high demands are placed on the biocompatibility of the ingredients for formulations that have a long application. In order to minimise adverse health effects for patients.

Formulations which have a continuous, controlled delivery over a long application and a high biocompatibility as well as good properties in respect of their biodegradability are therefore required.

WO 2005 102 284 discloses a formulation comprising a protein active substance, a biodegradable polymer, and a lipid, which is solid at room temperature.

WO 2004 011 054 describes an injectable depot form formulation comprising a plurality of biocompatible and biodegradable polymers and a substance to forming a gel, in which an active substance is dispersed.

The administration systems described in the prior art have short application times and might not even contain the necessary therapeutic active substance dose for the user.

The object of the present invention is therefore to provide a biodegradable depot form for parenteral administration of active substances which enables a continuing, controlled active substance release in a dose suitable for the therapy.

This object is achieved in accordance with the invention by an extruded depot form which comprises at least one active substance and at least two compounds of the class of substances which can be broken down by lipases, wherein the at least two compounds comprise a low-melting compound and a high-melting compound according to claim 1, and by a method for producing the depot form according to the invention according to claim 11. The object is also achieved by a composition according to the invention for use according to claim 15.

The present invention therefore relates to an extruded depot form for lasting active substance release comprising
a) at least one active substance,
b) at least two compounds of the class of substances which can be broken down by lipases, wherein the at least two compounds comprise a low-melting compound and a high-melting compound, and wherein the ratio of the low-melting compound to the high-melting compound ranges from 1:9 to 9:1, preferably ranges from 1:3 to 5:1, and
c) optionally at least one auxiliary agent for modulating the active substance release, wherein b), or optionally b) and c), constitute at least 60 wt. % of the dry weight of the depot form.

Extruded depot forms according to the invention in accordance with claim 1 advantageously have a controlled active substance delivery in a period of time of from one week to one year, a high biocompatibility, and a good biodegradability.

The term biodegradability shall be understood here to mean that substances contained in the formulation are broken down or eroded into smaller units in vivo, for example by enzymatic, chemical or physical processes.

In their simplest embodiments, the depot forms according to the invention comprise at least one active substance and at least two compounds of the class of substances which can be broken down by lipases, and optional auxiliary agents.

Here, the dry weight of the at least two compounds of the class of substances which can be broken down by lipases and optionally of the at least one contained auxiliary agent constitutes more than 60 wt. %, preferably more than 62 wt. %, particularly preferably 64 wt. %, in particular preferably at least 65 wt. % of the total weight of the depot form.

Here, the dry weight of the depot form denotes the weight of an administration-ready formulation which has no content or a negligible content of water, in particular less than 3 wt. %.

The total content of the at least two compounds of the class of substances which can be broken down by lipases here comprises the content in the active-substance-containing depot form of all compounds of the class of substances which can be broken down by lipases.

However, the dry weight of the at least two compounds of the class of substances which can be broken down by lipases and optionally of the at least one contained auxiliary constitutes at most a proportion of 99 wt. %, preferably at most 97.5 wt. %, particularly preferably at most 95 wt. %, in particular preferably at most 90 wt. % in the total weight of the depot form.

In a depot form according to the invention, a first compound of the class of substances which can be broken down by lipases has a lower melting point than a second compound of the class of substances which can be broken down by lipases, wherein the ratio of the at least two compounds to one another is an essential feature of the invention.

Hereinafter, the ratio of the at least two compounds of the class of substances which can be broken down by lipases to the ratio of the dry weight of a low-melting compound present in the depot form refers to the dry weight of a high-melting compound present in the depot form. In accordance with the invention the ratio of a low-melting compound to a high-melting compound ranges from approximately 1:9 to approximately 9:1, preferably ranges from approximately 1:6 to approximately 6:1, particularly preferably ranges from approximately 1:3 to approximately 5:1.

Hereinafter, a first compound from the class of substances which can be broken down by lipases having a lower melting point is referred to as a “low-melting compound”; accordingly, a second compound from the class of substances which can be broken down by lipases having a higher melting point is referred to as a “high-melting compound”.

The term “lipases” refers to enzymes which are able to cleave lipids. Lipases belong to the family of esterases and catalyse the hydrolysis of fats into fatty acids and glycerins. For example, lipoprotein lipase is relevant for the breakdown of subcutaneous depot forms.

A depot form according to the invention may optionally contain at least one auxiliary agent which can influence the active substance release from the depot form, the active substance stability, the plasma half-life and/or the bioavailability of the at least one active substance. A preferred auxiliary agent supports the controlled delivery of the at least one active substance from the depot form. In particular, an auxiliary agent of this kind contributes to a long-lasting active substance delivery, but without disadvantageous influencing the biocompatibility.

Alternatively or additionally, the addition of such an auxiliary agent can improve the stability of the active substance contained in the depot form. This is of particular benefit if the depot form is intended for a long-lasting application from a number of weeks up to a year.

In its subcutaneous application, the extruded depot form according to the invention delivers the at least one active substance from the active-substance-containing depot form to the surrounding tissue, wherein a considerable amount of the active substance is absorbed systemically. Insofar as the extruded depot form is intended for local therapy, a significant amount of the active substance can be delivered advantageously into the tissue surrounding the application site.

The absolute active substance amount contained in the depot form generally determine the period of time over which a continuous supply of the active substance into or to the organism is maintained. The highest possible loading of the depot form with at least one active substance is therefore desirable if the application time of the depot form is long, i.e. a number of weeks up to 12 months.

An extruded depot form according to the invention is preferably used for an application period from at least one week to at most 12 months, preferably for an application period from one week to 6 months, in particular for an application period from 2 weeks to 3 months.

The present invention therefore relates to the medical, veterinary and/or cosmetic use of the depot form according to the invention to deliver active substances into the bloodstream of a human or animal body.

The present invention also relates to a method for producing a depot form according to the invention, wherein the method comprises the following steps:
(i) providing a homogeneous mixture comprising at least one active substance, at least two compounds of the class of substances which can be broken down by lipases, wherein the at least two compounds comprise a low-melting compound and a high-melting compound, and wherein the ratio of the low-melting compound to the high-melting compound, and wherein the ratio of the low-melting compound to high-melting compound ranges from 1:9 to 9:1, preferably ranges from 1:3 to 5:1, and optionally at least one auxiliary agent for modulating the active substance release,
(ii) extruding the mixture, in particular by melt extrusion, to obtain the extrudate at a temperature below the melting point of the high-melting compound,
(iii) cutting the extrudate into pieces of suitable size,
(iv) optionally rounding the pieces, in particular by spheronisation,
(v) optionally applying a coating mixture or composition to the extrudate obtained above,
(vi) optionally carrying out a sterilisation process and/or packaging the depot form.

Here, provision shall be understood to mean an on-site production and also a delivery of a homogeneous mixture. A homogeneous mixture can be produced by a suitable mixing process, preferably without addition of solvents. The mixing process can additionally include more than one step, for example if firstly a mixture of the low-melting and the high-melting compound and, separately, a mixture of the at least one auxiliary agent and the at least one active substance are provided and are then mixed together in a second step.

The resultant homogeneous mixture is then heated to a temperature below the temperature of the melting point of the used high-melting compound and is then extruded by means of extrusion, in particular by means of melt extrusion, to obtain the extrudate or the core. Once the extrudate or the core has been cut into pieces of suitable size, a homogeneous coating mixture or composition consisting of at least one of the above-mentioned components (i) of the depot form according to the invention is applied optionally.

The components according to (i) advantageously comprise at least 60 wt. % of the dry weight of the depot form according to the invention, preferably at least 62 wt. %, particularly preferably at least 64 wt. %, in particular preferably at least 65 wt. % of the total weight of the depot form according to the invention.

The depot form according to the invention can therefore advantageously be produced at temperatures at which even most sensitive active substances can be processed without any adverse effects. This is of interest in particular for protein active substances and the like.

Insofar as the production has not already taken place under aseptic conditions, an advantageous production method can provide a sterilisation of the extruded depot form according to the invention prior to a possible packaging step. However, a depot form according to the invention can also be produced without a sterilisation process or also under conditions which are not aseptic.

Furthermore, the extruded depot form according to the invention can be subjected to a packaging process, in which the depot form is packaged directly into a packaging unit following a possible sterilisation process. Alternatively, the extruded depot form can also be introduced firstly into an applicator provided for application of the depot form according to the invention and can be packaged jointly therewith in a packaging unit.

Lastly, the present invention comprises an extruded depot form which is obtainable by a method as described above.

Further particularly advantageous embodiments and developments of the invention will become clear from the dependent claims in the following description, wherein the claims of each category can also be developed in accordance with the dependent claims of another category, and features of various exemplary embodiments can be combined to form new exemplary embodiments.

A preferred extruded depot form comprises a low-melting compound and a high-melting compound, wherein the melting point of a low-melting compound is at most 45° C., preferably at most 44° C., in particular less than 43° C. and/or the melting point of a high-melting compound is above 45° C., preferably at least 50° C., in particular at least 60° C. The low-melting compound particularly preferably has a melting point of from approximately 30 to approximately 43° C., and the high-melting compound has a melting point of from approximately 46 to approximately 75° C.

The difference of the melting points between the at least two compounds of the class of substances which can be broken down by lipases preferably varies in a range of from approximately 1 to approximately 45° C. The difference, however, in the melting points between the low-melting and the high-melting compounds is preferably approximately 2 to approximately 40° C., particularly preferably approximately 5 to approximately 35° C., in particular approximately 10 to approximately 32.5° C., in particular preferably approximately 15 to approximately 30° C.

In accordance with a preferred embodiment both the low-melting compound and the high-melting compound are selected from mono-, di- and/or triglycerides, for example esterifications of glycerin with saturated or unsaturated fatty acids with a length of from 5 to 20 carbon atoms, phosphatidic acid, lecithin, phosphatidylethanolamine, phosphatidylinositol, phosphatidylserine, phosphatidylglycerin, ceramides, cerebrosides, gangliosides, sphingophospholipids, sphingomyelins, sphingosulfatides, glycosphingosides, acylamino sugars, acylamino sugar glycans, acyltrehaloses, acyltrehaloseglycans, sorbitol fatty acid esters, squalene, steroids, polyketides, sterol lipids, prenol lipids, cholesterol, hard fats, waxes, and salts and derivatives thereof.

Examples of preferred low-melting compounds of the class of substances which can be broken down by lipases are hard fats which for example consist of a mixture of mono-, di- and triglycerides, which mixture can be obtained by esterifications of fatty acids of natural origin with glycerin or by re-esterification of fats of natural origin. Such hard fats are described in the Pharmacopoea Europaea (Ph. Eur. 8th edition, fundamental work 2014) and can be referred to for example by the name Witepsol E85, Witepsol H5, Witepsol H12, Witepsol H37 and/or Witepsol H15 by the company 101 Oleo GmbH (Germany).

Preferred high-melting compounds of the class of substances which can be broken down by lipases can be selected here from Dynasan 112, Dynasan 116 and/or Dynasan 118 These are obtainable for example from the company 101 Oleo GmbH (Germany).

Furthermore, extruded depot forms can also comprise more than two compounds of the class of substances which can be broken down by lipases. In such a case, the ratio of low-melting compound to high-melting compound relates to the dry weight fraction of all low-melting compounds to the dry weight fraction of all high-melting compounds. This ratio ranges from approximately 1:9 to approximately 9:1, particularly preferably ranges from approximately 1:6 to approximately 6:1, in particular ranges from approximately 1:3 to approximately 5:1. Extruded depot forms, however, preferably comprise merely than two compounds of the class of substances which can be broken down by lipases.

Exemplary combinations of at least two compounds of the class of substances which can be broken down by lipases are summarised in Table 1.

TABLE 1

| Low-melting | High-melting | |
|---|---|---|
| compound(s) | compound(s) | Weight ratio |
| 100 wt. % Witepsol E85 | 0 wt. % Dynasan 118 | 1:0 |
| 50 wt. % Witepsol H12 | 50 wt. % Dynasan 118 | 1:1 |
| 60 wt. % Witepsol H12 | 40 wt. % Dynasan 118 | 1.5:1 |
| 75 wt. % Witepsol H12 | 25 wt. % Dynasan 116 | 3:1 |
| 30 wt. % Witepsol E85 | 70 wt. % Dynasan 118 | 3:7 |
| 80 wt. % Witepsol E85 | 20 wt. % Dynasan 112 | 4:1 |
| 40 wt. % Witepsol H12, | 40 wt. % Dynasan 118 | 1.5:1 |
| 20 wt. % Witepsol E85 | | |
| 25 wt. % Witepsol E85 | 20 wt. % Dynasan 112, | 1:3 |
| | 55 wt. % Dynasan 118 | |
| 15 wt. % Witepsol H12, | 40 wt. % Dynasan 116, | 1:2.33 |
| 15 wt. % Witepsol E85 | 30 wt. % Dynasan 118 | |

At least one active substance is contained in the depot forms according to the invention. This at least one active substance, without limitation, is selected from the class of antibiotics, antimicrobiotics, antimycotics, antiseptics, chemotherapeutics, cytostatics, metastasis inhibitors, antiallergics, anticoagulants, sexual hormones, sexual hormone inhibitors, haemostyptics, hormones, peptide hormones, antidepressants, vaccines, gonadotropin-releasing hormone analogues, growth factor inhibitors, hormone mimetics, multiple sclerosis therapeutics, programmed cell death receptor antagonists, neuroleptics, complement system inhibitors, vitamins, antihistamines, antibodies, DNA, plasmid DNA, cationic DNA complexes and RNA (such as siRNA or mRNA), fusion proteins and anti-diabetics.

Useful active substances include, without limitation, Heparin, heparin derivatives, Hirudin, acetylsalicylic acid, Enoxaparin, Liraglutide, Albiglutide, Dulaglutide, Lixisenatide, Exenatide, Insulin, Insulin analogues, Acarbose, Glatirameracetate, Octreotide, Desmopressin, Oxytocin, Zafirlukast, Buserelin, Somatostatin, Glibenclamide, Gliclazide, Glimepiride, Gliquidone, Pioglitazone, Miglitol, Nateglinidee, Mitiglinid, Repaglinide, Sitagliptin, Vildagliptin, Dexamethasone, Prednisolone, Corticosterone, Budesonide, Oestrogen, Sulfasalazine, Mesalazine, Risperidone, Paclitaxel, 5-Fluoruracil, Cisplatin, Vinblastine, Vincristine, Epothilone, Endostatine, Angiostatin, D-Phe-Pro-Arg-Chloromethylketone and monoclonal antibodies, such as Adalimumab, Aducanumab, Aflibercept, Benralizumab, Bevacizumab, Blinatumomab, Certolizumab, Denosumab, Dupilumab, Efalizumab, Erenumab, Infliximab, Ipilimumab, Mepolizumab, Natalizumab, Nemolizumab, Ocrelizumab, Omalizumab, Pembrolizumab, Pertuzumab, Ranibizumab, Reslizumab, Rituximab, Solanezumab, Tocilizumab, Tralokinumab, Trastuzumab, Ustekinumab and Vedolizumab.

Preferred extruded depot forms containing at least one active substance can be used as for therapy of cancer diseases, for example multiple myeloma, mantle cell lymphoma, diffuse large cell B-cell lymphoma, acute myeloid lymphoma, follicular lymphoma, chronic lymphocyte leukaemia, breast, lung, endometrial, ovarian, stomach, cervical or prostate cancer, pancreatic carcinoma, glioblastoma kidney carcinoma, hepatocellular carcinoma, colon carcinoma, neuroendocrine tumours head and neck tumours, sarcoma, tumour syndrome is resulting directly or indirectly from genetic defects in tumour suppressor organs such as P53, PTEN or VHL, endometrial carcinoma, lymphangioleiomyomatosis, neurofibromatosis 1, Hippel Lindau syndrome, and rheumatoid arthritis, spondylitis ankylosans (morbus bechterew), psoriasis arthritis, psoriasis, osteoarthritis, gout, asthma, bronchitis, allergic rhinitis, chronic obstructive pulmonary disease, cystic fibrosis, chronic intestinal sensitivity disorders, irritable bowel, mucous colitis, ulcerative colitis, Crohn's disease, chorea huntington's disease, gastritis, oesophagitis, hepatitis, pancreatitis, nephritis, lupus erythematodes, atherosclerosis, restenosis following angioplasy, left-ventricular hypertrophy, myocardial infarction, heart attack, ischaemic damage to the heart, lungs, intestine, kidneys, liver, pancreas, spleen and brain, acute or chronic organ transplant rejection, macular degeneration, diabetic macular oedema, anaemia, fertility disorders, obesity, Pubertas praecox, endometriosis, mastodynia, Tourette's syndrome, depression, personality disorders, compulsive disorders, ADHS in children, irritability in foetal alcohol syndrome and autism, and delusions, hallucinations, epilepsy, Alzheimer's disease, Parkinson's disease, paroxysmal nocturnal haemoglobinuria, as sedatives, for gender reassignment measures, multiple sclerosis and diabetes.

In accordance with a preferred embodiment and advantageous extruded depot form contains at least one active substance of the class of monoclonal antibodies, neuroleptics or anti-diabetics, more preferably of the class of type I anti-diabetics and/or type 2 anti-diabetic, in particular preferably the class of incretin mimetics.

In accordance with a particularly preferred embodiment the at least one active substance is selected from bevacizumab, ranibizumab, citalopram, risperidon, insulin and/or glucagon-like peptidel (GLP-1) analogues, such as Liraglutide, Albiglutide, Dulaglutide, Lixisenatide and/or Exenatide, in particular from Bevacizumab, Risperidon or Exenatide, in particular preferably from Bevacizumab and/or Exenatide.

In particular, preferred depot forms are used for the treatment of macular degeneration or of diabetes mellitus, particularly preferably the treatment of type I diabetes mellitus and type II diabetes mellitus, in particular preferably for the treatment of type II diabetes mellitus.

The active substance of Exenatide, which is preferred in particular, may advantageously comprise a polypeptide form from 39 amino acids with the following sequence:

His Gly Glu Gly Thr Phe Thr Ser Asp Leu Ser Lys
Gln Met Glu Glu Glu Ala Val Arg Leu Phe Ile Glu
Trp Leu Lys Asn Gly Gly Pro Ser Ser Gly Ala Pro
Pro Pro Ser

The at least one active substance can also be contained in the depot form in various forms, depending on which form results in an optimal release property of the active substance from the depot form. Amino acid-based active substances can be present generally as cyclopeptide, oligopeptide or polypeptide or other pharmacologically acceptable derivatives or as components of molecular complexes. The amino acids can be linked to one another both via a peptide bonds and via w peptide bonds. The at least one active substance can also be present in the form of a salt, for example acetate, or also in the form of the free base or acid.

Furthermore, at least one of the amino acids of the amino acid-based active substances mentioned above as preferred active substances can contain post-translational modifications. These post-translational modifications advantageously do not influence the properties of the active substance, in particular in respect of the release and effect.

In principle, the content of the active substance in the depot form according to the invention can vary within a wide range. An advantageous active substance amount, preferably of monoclonal antibodies, growth factor inhibitors or anti-diabetics, particularly preferably of VEGF inhibitors or anti-diabetics of the class of incretin mimetics, in particular of Bevacizumab, Ranibizumab or Exenatide, in particular preferably of Bevacizumab or Exenatide, is approximately 0.3 wt. % to approximately 50 wt. %, preferably approximately 3 wt. % to approximately 30 wt. %, particularly preferably approximately 4 wt. % to approximately 25 wt. %, in particular approximately 7 wt. % to approximately 20 wt. %.

For effective therapy the active substance concentration of the active substance that is found in the blood of the user following administration of a depot form is significant. A substantially constant plasma concentration of the active substance of at least 50 pg/mL over a period of time of at least one week up to at most 12 months, preferably from at least one week to 6 months, can therefore advantageously be attained with the aid of an extruded depot form which contains an incretin mimetic, in particular Exenatide.

Extruded depot forms according to the invention are also suitable for cosmetic applications. In particular, an advantageous depot form can be used for cosmetic wrinkle smoothing. The composition according to the invention is used for local, in particular targeted, wrinkle smoothing, in particular preferably for preventing wrinkles, for skin firming and for protection against skin ageing. Exemplary active substances for this purpose can be selected from hyaluronic acid, collagen and/or botox.

Advantageous depot forms can additionally contain at least one auxiliary agent which is conventional for subcutaneous application forms and which modulates the active substance release. Reference can be made here in particular to substances that are used in the production of subcutaneous implants and that are physiologically safe. The auxiliary agents preferably have a high biocompatibility, and therefore the auxiliary agents and any degradation products of the auxiliaries agents are not toxic for the user and do not result in any undesirable side-effects.

It has been found that the addition of pore formers can advantageously improve the delivery of the at least one active substance from the subcutaneous depot form. A pore former of this kind can be selected for example from the group of hydrophilic substances, such as calcium sulphate, calcium hydrogen phosphate, sugars such as glucose, lactose, fructose, mannitol, trehalose, dextrins, maltodextrin, sucrose, sorbitol, xylitol, starch and derivatives thereof, for example hydroxyethyl starch, polyvinylpyrrolidone, polyethylene glycol such as PEG 6000 or PEG 8000, sodium chloride, sodium citrate, citric acid, hyaluronic acid, polyvinyl alcohols, polyacrylic acid and derivatives thereof, polymethacrylic acid and derivatives thereof, polymethyl methacrylate, polystyrene, copolymers with monomers of methyl methacrylate and styrene, and mixtures thereof.

Suitable particularly preferred pore formers are trehalose and/or hydroxyethyl starch and/or polyethylene glycol (PEG), which for example can be procured from the company Clariant or Sigma-Aldrich (Austria).

The molecular weight of a pore former, in particular of PEG, preferably ranges from approximately 1 to approximately 20 kDa. The molecular weight particularly preferably ranges from approximately 3 to approximately 10 kDa, in particular ranges from approximately 4 to approximately 8 kDa.

It has also been found that the delivery of the at least one active substance from the depot form according to the invention can be improved if organic substances based on lactic acid are contained in the depot form, for example poly(L-lactide), poly(D,L-lactide), poly(glycolide), poly(L-lactide-co-D,L-lactide), poly(L-lactide-co-glycolide), poly(D,L-lactide-co-glycolide), poly(meso-lactide), poly(D,L-lactide-co-trimethylenecarbonate), poly(trimethylenecarbonate), poly(epsilon-caprolactone), poly(L-lactide-co-meso-lactide), poly(L-lactide-co-epsilon-caprolactone), poly(D,L-lactide-co-meso-lactide), poly(D,L-lactide-co-epsilon-caprolactone), poly(meso-lactide-co-glycolide), poly(meso-lactide-co-trimethylenecarbonate), poly(meso-lactide-co-epsilon-caprolactone), poly(glycolide-co-trimethylenecarbonate), poly(glycolide-co-epsilon-caprolactone), poly(phosphazene), poly(glycolide-co-caprolactone) and the like. Poly(D,L-lactide) and poly(D,L-lactide-co-glycolide) (PLGA), which are obtainable for example from the company Evonik Industries AG (Germany) under the names R 202 H (Poly(D,L-lactide)) and RG 502 H and RG 752 H (PLGA) are suitable as a particularly preferred release modulators.

If a release modulator, in particular poly(D,L-lactide) or PLGA, is contained in the depot form, the molecular weight thereof can vary in principle within a wide range. The molecular weight, however, preferably ranges from approximately 5 to approximately 100 kDa. The molecular weight particularly preferably ranges from approximately 7 to approximately 60 kDa, in particular ranges from approximately 9 to approximately 40 kDa.

The active substance delivery rate can also be increased by addition of a swellable polymer, which preferably are selected from collagen, gelatin and derivatives thereof, starch and derivatives thereof (preferably hydroxyethyl starch, hydroxypropyl starch, carboxymethyl starch), cellulose derivatives, chitin, chitosan and derivatives thereof, polyamides, polyhydroxy acids, polyhydroxybutyrates, polyhydroxyvalerates, polycaprolactones and polydioxanones. This is relevant in particular for active substances for which a higher dose is required and/or active-substance-containing depot forms with a shorter application period, for example an application period of from a few weeks to a few months. A particularly preferred swellable polymer in this context is hydroxyethyl starch (HES) and can be procured from Sigma Aldrich (Austria).

The molecular weight of a swellable polymer, in particular of HES, advantageously ranges from approximately 50 to approximately 400 kDa. The molecular weight particularly preferably ranges from approximately 90 to approximately 300 kDa, in particular ranges from approximately 130 to approximately 200 kDa. The degree of substitution of HES, that is to say the ratio of the number of glucose units modified with hydroxyalkyl groups to the total number of monomer units, ranges here from approximately 0.1 to approximately 1.

In addition, the depot forms according to the invention can also contain further conventional auxiliary agents known to a person skilled in the art, for example tocopherols, for example α-tocopherol, β-tocopherol, γ-tocopherol, δ-tocopherol and mixtures thereof (vitamin E) which in particular are used as antioxidants.

In an advantageous embodiment an antioxidant of this kind inactivates reactive oxygen species in the depot form, whereby oxidation of the active substance is slowed or completely prevented, and thus improves the stability of the active substance and thus provides a longer shelf life of the depot form according to the invention, both during storage and during use.

A content of one or more of the preferred auxiliary agents advantageously results in a controlled and continuing active substance release from the preferred extruded depot form.

Advantageous compositions of an extruded depot form are presented as follows:
(i) 0.1 to 50 wt. % active substance, preferably 1 to 30 wt. %, in particular 2.5 to 25 wt. % active substance,
(ii) 1 to 95 wt. % of a low-melting compound, preferably 10 to 89 wt. % of a low-melting compound,
(iii) 1 to 89 wt. % of a high-melting compound, preferably 5 to 60 wt. % of a high-melting compound,
(iv) optionally 1 to 25 wt. % trehalose, preferably 5 to 20 wt. % trehalose, (v) optionally 1 to 79 wt. % PLGA, preferably 5 to 65 wt. % PLGA, and
(vi) optionally 1 to 25 wt. % PEG, preferably 5 to 20 wt. % PEG.

A composition of an extruded depot form particularly preferably comprises
(i) 3 to 25 wt. % active substance, preferably 4 to 20 wt. % active substance,
(ii) 30 to 85 wt. % of a low-melting compound,
(iii) 10 to 58 wt. % of a high-melting compound,
(iv) optionally 2.5 to 18 wt. % trehalose, preferably 3 to 16 wt. % trehalose, in particular 3.5 to 15 wt. % trehalose,
(v) optionally 7.5 to 60 wt. % PLGA, and
(vi) optionally 3 to 17.5 wt. % PEG.

The constituents of an extruded depot form are in particular preferably selected from
(i) 5 to 15 wt. % active substance,
(ii) 40 to 75 wt. % of a low-melting compound,
(iii) 15 to 55 wt. % of a high-melting compound,
(iv) optionally 5 to 10 wt. % trehalose, and
(v) optionally 10 to 40 wt. % PLGA, and
(vi) optionally 5 to 15 wt. % PEG.

In particular, an advantageous active substance content is approximately 6 to 14 wt. %, a content of a low-melting compound is approximately 42 to 60 wt. %, and a content of a high-melting compound is approximately 40 wt. %.

In accordance with a further preferred embodiment trehalose can be replaced fully or partially by HES.

As mentioned at the outset, extruded depot forms according to the invention are produced by means of extrusion. It has been found that the properties of the mixture, prepared for extrusion, of the at least two compounds of the class of substances which can be broken down by lipases with the at least one active substance improve if the active substance is admixed in the form of dried powder, preferably in the form of spray-dried or freeze-dried powder or lyophilisate, to the at least two compounds of the class of substances which can be broken down by lipases.

Insofar as active substances are used in a dissolved state, a drying step, in particular a lyophilisation step, is preferably performed prior to the mixing of the substances for producing the depot form according to the invention.

In principle, a large number of substances can be added to the active substance for this purpose so as to advantageously obtain the bioactivity of the active substance. Such substances are also referred to as cryoprotectors or lyoprotectors, wherein lyoprotectors in this regard are used to protect the substances during a drying process and cryoprotectors have a corresponding task during a freezing process.

It has also been found that the length of the active substance release following the subcutaneous application can be influences by not cooling the extrudate directly after extrusion to ambient temperature, but instead are stored for example in a drying cabinet or incubator for a certain time at increased temperature. A period of time ranging from approximately 0.5 to approximately 5 hours has proven to be advantageous in this regard. The optional storage at elevated temperature, also referred to as a temperature-control step, is coordinated with the melting point of the low-melting lipid and for example ranges from approximately 30 to approximately 60° C., preferably approximately 35 to approximately 50° C., particularly preferably approximately 37 to 47° C.

Preferred depot forms can additionally be produced by a rounding method, in particular by spheronisation. The cylindrical extrudate is advantageously rounded such that any forms, for example corners and edges, resulting from the extrusion and which might have a disadvantageous effect on the application properties are removed. The spheronisation can additionally be used for the production of microparticles, which are then administered subcutaneously and thus likewise represent a biodegradable depot form.

A depot form advantageously has a homogeneous coating which consists of at least one layer applied to the core and advantageously delimits the initial release of active substance from the depot form and ensures therapeutic concentrations of the at least one active substance over a prolonged period of time. A preferred coating comprises a mixture of substances or a composition which are/is selected from at least one of the above-mentioned components a) to c) of the depot form according to the invention, and for example can thus also be provided free from active substance. If the coating contains an active substance, the content thereof can be the same as or different from the active substance content of the core. In a particularly preferred embodiment, however, the coating is free from active substance.

In principle, a suitable weight of an extruded depot form varies within a range that is conventional for subcutaneous implants. The weight of the extruded depot form is also dependent on the desired application time and/or the application site. A preferred weight of an extruded depot form, however, ranges from 1 to 1000 mg, particularly preferably ranges from 5 to 100 mg, in particular ranges from 7.5 to 75 mg, in particular preferably ranges from 10 to 60 mg.

A depot form in the sense of the invention can be embodied in the form of rods, balls, cubes, ellipsoids, cuboids, pillows, cylinders, tablets, pellets, platelets, or briquettes.

Depot forms according to the invention are preferably of injectable size, but, if desirable, can also be introduced to the administration site by means of a surgical intervention. Preferred depot forms have a diameter of at least 01 to 10 mm and a length of at least 0.15 to 50 mm, in particular, depot forms have a diameter of at least 0.15 to 7.5 mm and a length of at least 0.2 to 45 mm, in particular preferably a diameter of at least 0.2 to 5 mm and a length of at least 0.3 to 40 mm.

Insofar as extruded depot forms are provided in the form of microparticles, the diameter of the round or practically round particles can be approximately 1 to approximately 100 μm, preferably approximately 5 to approximately 90 μm, in particular preferably approximately 10 to approximately 80 μm.

The term “diameter” refers here to the longest path running orthogonally to an axis of rotation and connecting two points of the edge of the body in question to one another. The straight line about which a rotation body can be rotated denoted the axis of rotation.

The term “length” relates to a part of the axis of rotation that is disposed within the rotation body.

The ratio of diameter to length of preferred depot forms ranges advantageously from 1:30 to 10:1, preferably ranges from 1:15 to 5:1, in particular preferably ranges from 1:13.3 to 1:1.

As already explained, a method according to the invention for producing the extruded depot form comprises the mixing of (a) the at least one active substance, (b) the at least two compounds of the class of substances which can be broken down by lipases, and optionally (c) the at least one auxiliary agent, whereby a homogeneous powder mixture is obtained.

In accordance with a further preferred method, the extruded depot form is produced with the aid of what is known as double extrusion, which is characterised in that. after the first extrusion of the mixture according to step (ii) of the above-explained method, a second extrusion is performed.

Such a preferred method comprises the following steps:
(i) providing a homogeneous mixture comprising at least one active substance, at least two compounds of the class of substances which can be broken down by lipases, wherein the at least two compounds comprise a low-melting compound and a high-melting compound, and wherein the ratio of the low-melting compound to the high-melting compound, and wherein the ratio of the low-melting compound to high-melting compound ranges from 1:9 to 9:1, preferably ranges from 1:3 to 5:1, and optionally at least one auxiliary agent for modulating the active substance release,
(ii) extruding the mixture, in particular by melt extrusion, to obtain the extrudate at a temperature below the melting point of the high-melting compound,
(iii) optionally cutting the extrudate from step (ii) into pieces of suitable size,
(iv) extruding the mixture, in particular by melt extrusion, at a temperature below the melting point of the high-melting compound,
(v) cutting the extrudate into pieces of suitable size,
(vi) optionally rounding the pieces, in particular by spheronisation,
(vii) optionally applying the coating mixture to the extrudate obtained above,
(ix) optionally carrying out a sterilisation process and/or packaging the depot form.

Insofar as an extruded depot form is produced by such a method, the diameter of the depot form provided for use is set advantageously during the course of the second extrusion, whereby an improved homogeneity of the extrudate can be obtained.

A first and/or a second extrusion can be performed here with the aid of a screw extruder, as obtainable for example under the name Mini CTW from the company Thermo Fisher Scientific GmbH (Karlsruhe, Germany). Co-rotating or counter-rotating screws can be used advantageously for a first and/or a second extrusion. Co-rotating screws are particularly preferably used for a first extrusion, and counter-rotating screws are particularly preferably used for a second extrusion.

The extrudate can be cut after the first extrusion. Following the second extrusion or after the double extrusion, the extrudate Is advantageously cut into implants or pieces of suitable length or is further processed by a comminution, for example with the aid of a homogeniser or a cryo-grinding process, to form microparticles.

A powder mixture in the sense of the present invention is understood here to mean a mixture of a number of solid constituents of suitable size, wherein the constituents can comprise particles of a size smaller than 1 nm. A powder mixture can also comprise particles with a size that ranges from 1 nm to 1 μm and/or particles with a size bigger than 1 μm. If at least one of the constituents to be mixed is not present in solid form prior to the mixing, it can be converted into the solid state, for example by spray-drying or freeze-drying, before the powder mixture is created.

For application of the extruded depot form according to the invention into the subcutaneous tissue, all application devices known to a person skilled in the art can be used in principle. Depot forms according to the invention can thus be administered for example by syringes, cannulas, applicators and injectors, in particular by applicators.

Lastly, the present invention also relates to a kit comprising an extruded depot form according to the invention and an applicator suitable for application, by means of which the depot form can be administered subcutaneously. Extruded depot forms according to the invention do not necessarily have to be sterilised before being introduced into the applicator or do not necessarily have to be produced generally under aseptic conditions, and instead can also be subjected to a sterilisation process within the applicator in the end container. An applicator of this kind is also able to receive extruded depot forms of different length. Of course, depot forms which have a cylindrical shape, but for example are square or round or the like, can also be applied.

Such an applicator expediently has a hollow needle for receiving extruded depot forms having the above-explained dimensions and has a protective cap which is to be removed prior to use and can be fixed in place again after use. The extruded depot form is thus advantageously protected against external influences which might in some way negatively influence the preferred use.

Further features of the invention will become clear from the following description of exemplary embodiments in conjunction with the claims and the drawings. It should be noted that the invention is not limited to the embodiments of the described examples, but is specified by the scope of the accompanying claims. In particular, the individual features in embodiments according to the invention can be implemented in combinations other than those presented in the examples described below. The following explanation of some exemplary embodiments of the invention is provided with reference to the accompanying drawings. In the drawings:

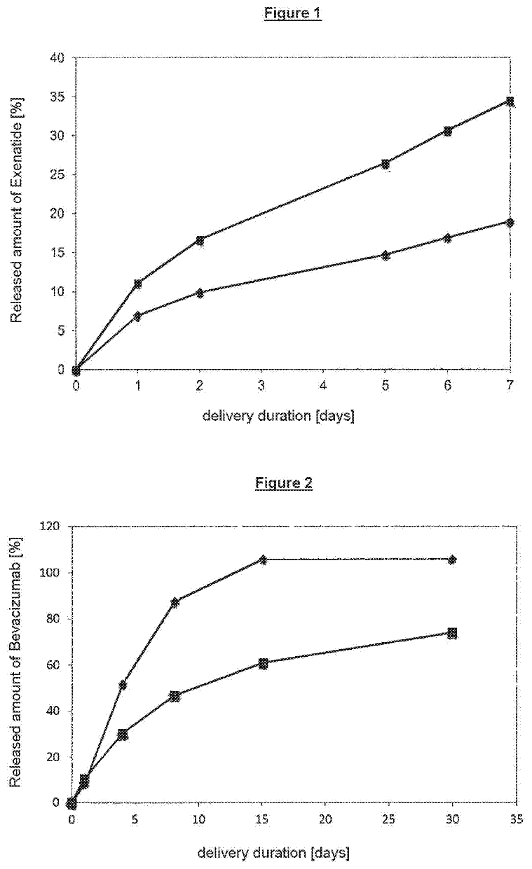

FIG. 1 shows release profiles of depot forms according to the invention with different exenatide-containing formulations and with a diameter of approximately 1.5 mm. Solid line: 10 wt. % Exenatide; Witepsol E85/Dynasan 118=35:65. Dashed line: 5 wt. % Exenatide; 5 wt. % PEG8000; Witepsol E85/Dynasan 118=35:65.

FIG. 2 shows release profiles of depot forms according to the invention containing Bevacizumab and having a diameter of approximately 1.9 mm. Solid line: 10 wt. % Bevacizumab; 10 wt. % PEG 6000; Witepsol H12/Dynasan 118=30:70. Dashed line: 10 wt. % Bevacizumab; 10 wt. % PEG 6000; Dynasan 112/Dynasan 118=30:70.

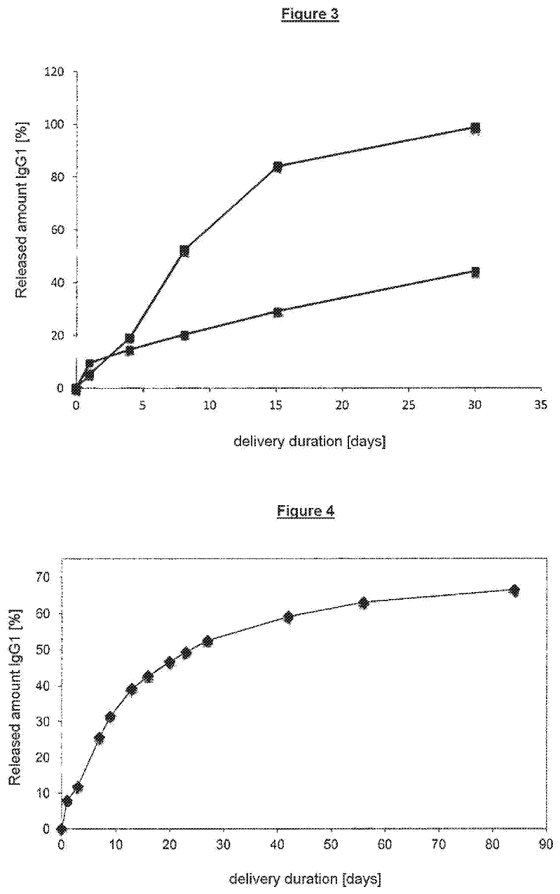

FIG. 3 shows release profiles of depot forms according to the invention containing IgG1 antibodies and having a diameter of approximately 1.9 mm. Solid line: 5 wt. % IgG1; 10 wt. % PEG 6000; Witepsol H12/Dynasan 118=30:70. Dashed line: 5 wt. % IgG1; 10 wt. % PEG 6000; Dynasan 112/Dynasan 118=30:70.

FIG. 4 shows a release profile of a depot form according to the invention containing IgG1 antibodies and having a diameter of approximately 1.5 mm and a length of 2 cm. Solid line: 10 wt. % IgG1 lyophilisate; Witepsol E85/Dynasan 118=50:50.

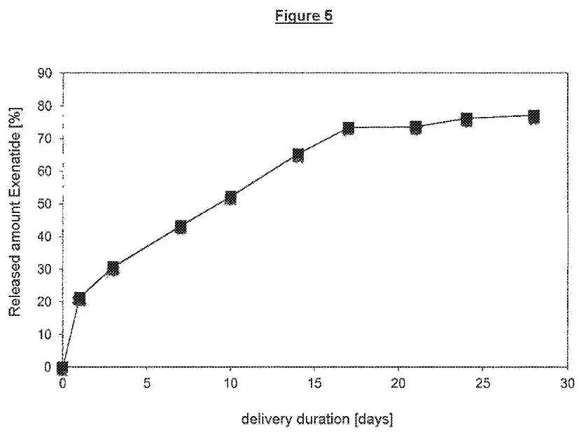

FIG. 5 shows a release profile of a depot form according to the invention containing Exenatide. The depot form was produced with the aid of a double extrusion, has a diameter of 1.2 mm, and a length of 1.5 cm. The depot form contains 10 wt. % Exenatide lyophilisate and E85/Dynasan 118=60/40.

### EXAMPLES

### Example 1

For the production of the depot forms according to the invention, lipid pellets formed from a low-melting hard fat (Witepsol E85, 101 Oleo GmbH, Hamburg, Germany) and a high-melting triglyceride (Dynasan 118, 101 Oleo GmbH, Hamburg, Germany) were cryo-ground separately (Freezer/Mill, C3 Prozess- and Analysentechnik GmbH, Haar bei München, Germany) and then 45 wt. % of the high-melting triglyceride and 45 wt. % of the low-melting hard fat were mixed with 10 wt. % Exenatide lyophilisate (Bachem, Bubendorf, Switzerland) to form a homogeneous mixture (Speedmixer, Hauschild, Hamm, Germany).

The following extrusion was carried out by way of co-rotating screw melt extrusion (Mini CTW, Thermo Fisher Scientific GmbH, Karlsruhe, Germany) at 35 to 42° C. and a screw speed of 40 rpm. The diameter for the extrudate was set using a nozzle to 1.5 mm. Optionally, a temperature-controlled step can be performed here, the temperature of which is coordinated with the melting point of the low-melting lipid and is approximately 35 to 45° C. The extrudate strand was cut to form extrudates of suitable length. Alternatively, the extrudate could be shaped by spheronisation to form microparticles.

### Example 2

The production was performed in accordance with Example 1, however the composition of the depot forms according to the invention was supplemented with trehalose (Sigma Aldrich, Vienna, Austria). The powder mixture consisted of 40 wt. % of a high-melting triglyceride (Dynasan 118, 101 Oleo GmbH, Hamburg, Germany), 40 wt. % of a low-melting hard fat (Witepsol E85, 101 Oleo GmbH, Hamburg, Germany), 10 wt. % trehalose, and 10 wt. % Exenatide lyophilisate (Bachem, Bubendorf, Switzerland).

### Example 3

The production was performed again in accordance with Example 1, wherein, in addition, PLGA was incorporated into the matrix. The powder mixture consisted of 35 wt. % of a high-melting triglyceride (Dynasan 118, IOI Oleo GmbH, Hamburg, Germany), 35 wt. % of a low-melting hard fat (Witepsol E85, IOI Oleo GmbH, Hamburg, Germany), 20 wt. % PLGA (Evonik Industries AG, Essen, Germany), and 10 wt. % Exenatide lyophilisate (Bachem, Bubendorf, Switzerland).

In order to examine the active substance release, exemplary depot forms according to the invention in the suitable size and shape (for example cut into cylinders of from 1.5 to 2 cm length) were firstly weighed individually.

The depot forms according to the invention were introduced into release cells and were mixed with 25 mL release medium (50 mM phosphate buffer). At the time at which the sample was taken, the release medium was replaced in full. The release rates were determined by UV-metric analysis.

### Example 4

Lipid pellets formed from Witepsol H12 and Dynasan 118 in a ratio 1:1 were cryo-ground for the production of depot forms according to the invention. 90 wt. % of the resultant powder mixture was then provided with 10% Bevacizumab lyophilisate (for example lyophilised Avastin®, Roche, Basel, Switzerland) and processed to form a homogeneous mixture.

In order to examine the active substance release, exemplary depot forms according to the invention in the suitable size and shape (for example cut into cylinders of from 1.5 to 2 cm length) were firstly weighed individually.

The depot forms according to the invention were introduced into 2 mL Eppendorf tubes, and the release was analysed using a horizontal shaker (40 rpm) at 37° C. in PBS buffer (pH 7.4). Samples were taken at the measurement times. The active substance content was then determined at 280 nm using a UV vis spectrometer (Agilent, Boblingen, Germany).

### Example 5

For the production of the depot forms according to the invention, lipid pellets formed from a low-melting hard fat (Witepsol E85, 101 Oleo GmbH, Hamburg, Germany) and a high-melting triglyceride (Dynasan 118, 101 Oleo GmbH, Hamburg, Germany) were cryo-ground separately (Freezer/Mill, C3 Prozess- and Analysentechnik GmbH, Haar bei München, Germany) and then 36 wt. % of the high-melting triglyceride and 54 wt. % of the low-melting hard fat were mixed with 10 wt. % Exenatide lyophilisate (Bachem, Bubendorf, Switzerland) to form a homogeneous mixture (Speedmixer, Hauschild, Hamm, Germany).

The following extrusion was carried out by way of counter-rotating screw melt extrusion (Mini CTW, Thermo Fisher Scientific GmbH, Karlsruhe, Germany) at 38 to 40° C. and a screw speed of 20 rpm. In a next step exclusion was performed a second time under the same conditions. The diameter for the extrudate was set using a nozzle to 1.5 mm. Optionally, a temperature-controlled step can be performed here, the temperature of which is coordinated with the melting point of the low-melting lipid and is approximately 40 to 45° C. The extrudate strand was cut to form extrudates of suitable length. Alternatively, the extrudate could be shaped by spheronisation to form microparticles.

### Example 6

For the production of the depot forms according to the invention, lipid pellets formed from a low-melting hard fat (Witepsol E85, 101 Oleo GmbH, Hamburg, Germany) and a high-melting triglyceride (Dynasan 118, 101 Oleo GmbH, Hamburg, Germany) were cryo-ground separately (Freezer/Mill, C3 Prozess- and Analysentechnik GmbH, Haar bei München, Germany) and then 42.5 wt. % of the high-melting triglyceride and 42.5 wt. % of the low-melting hard fat were mixed with 15 wt. % Octreotide lyophilisate (Chemi S.P.A., Mailand, Italy) to form a homogeneous mixture (Speedmixer, Hauschild, Hamm, Germany).

The following extrusion was carried out by way of counter-rotating screw melt extrusion (Mini CTW, Thermo Fisher Scientific GmbH, Karlsruhe, Germany) at 40 to 42° C. and a screw speed of 20 rpm. In a next step exclusion was performed a second time under the same conditions. The diameter for the extrudate was set using a nozzle to 1.5 mm. Optionally, a temperature-controlled step can be performed here, the temperature of which is coordinated with the melting point of the low-melting lipid and is approximately 40 to 45° C. The extrudate strand was cut to form extrudates of suitable length. Alternatively, the extrudate could be shaped by spheronisation to form microparticles.

1. An extruded depot form, for continuing active substance release, comprising
(a) at least one active substance,
(b) at least two compounds of the class of substances which can be broken down by lipases, wherein the at least two compounds comprise a low-melting compound and a high-melting compound, and wherein the ratio of the low-melting compound to the high-melting compound ranges from 1:9 to 9:1, preferably ranges from 1:3 to 5:1, and
(c) optionally at least one auxiliary agent for modulating the active substance release, wherein b), or b) and c), constitute at least 60 wt. % of the dry weight of the depot form.

2. The extruded depot form according to claim 1, wherein the melting point of a first compound which can be broken down by lipase is at most 45° C. and/or the melting point of a second compound which can be broken down by lipases is above 45° C.

3. The extruded depot form according to claim 1, wherein both the low-melting compound and the high-melting compound are selected from a fat, a hard fat, a mono-, di- and/or triglyceride and salts thereof.

4. The extruded depot form according to claim 1, wherein the at least two compounds of the class of substances which can be broken down by lipases are selected from esterifications of glycerin with saturated and unsaturated fatty acids of a length of from 5 to 20 carbon atoms.

5. The extruded depot form according to claim 1, wherein the at least one auxiliary agent for modulating the active substance release is selected from starch, cellulose, trehalose, dextrin, poly-(D,L-lactide-co-glycolide), polyethylene glycol, hydroxyethyl starch, trehalose, polyethylene glycol and/or poly-(D,L-lactide-co-glycolide).

6. The extruded depot form according to claim 1, wherein the at least one active substance is selected from the group consisting of VEGF inhibitors, growth factor inhibitors, kinase inhibitors, cytostatics, vaccines, monoclonal antibodies, peptide hormones, fusion proteins, anticoagulants, growth hormones, gonadotropin-releasing hormone analogues, breast cancer therapeutic agents, multiple sclerosis therapeutic agents, programmed cell death receptor 1 antagonists, neuroleptics, protein drugs against paroxysmal nocturnal haemoglobinuria, anti-diabetics, antidepressants, Bevacizumab, Ranibizumab, Citalopram, Risperidon, Octreotide, Insulin, Glucagon-like peptide 1 (GLP-1) analogues, Liraglutide, Albiglutide, Dulaglutide, Lixisenatide and Exenatide.

7. The extruded depot form according to claim 1, wherein the extruded depot form is a homogeneous mixture.

8. The extruded depot form according to claim 1 comprising a homogeneous core coating which comprises the components a), b) and/or c).

9. The extruded depot form according to claim 1, wherein the extruded depot form has a length ranging from 0.1 cm to 5 cm.

10. The extruded depot form according to claim 1, wherein the extruded depot form has a ratio of diameter to length of from approximately 1:30 to approximately 10:1.

11. A method for producing the extruded depot form according to claim 1, comprising the steps of
(i) providing at least one homogeneous mixture comprising (a), (b) and optionally (c),
(ii) extruding the mixture, to obtain the extrudate at a temperature below the melting point of the high-melting compound,
(iii) cutting the extrudate into pieces of suitable size,
(iv) optionally rounding the pieces,
(v) optionally applying the coating mixture to the extrudate obtained above,
(vi) optionally carrying out a sterilisation process and/or packaging the depot form.

12. The method for producing an extruded depot form according to claim 11, wherein the method, after step (ii), comprises the following steps:
(iii) optionally cutting the extrudate from step (ii) into pieces of suitable size,
(viii) extruding the mixture, at a temperature below the melting point of the high-melting compound,
(ix) cutting the extrudate into pieces of suitable size,
(x) optionally rounding the pieces,
(xi) optionally applying the coating mixture to the extrudate obtained above,
(ix) optionally carrying out a sterilisation process and/or packaging the depot form.

13. (canceled)

14. The extruded depot form according to claim 1, wherein, following one-time addition of an incretin mimetic, a prolonged plasma concentration of the active substance of at least 50 pg/ml over a period of time from at least a week to at most 12 months, is retained.

15. (canceled)

16. A method of treatment of a condition by providing a pharmaceutically effective amount of the extended depot form according to claim 1, wherein the condition includes cancer diseases, retroviral infections, dementia, Alzheimer's disease, arteriosclerosis, bronchial asthma, hypertonia, COPD, hepatitis, osteoporosis, coronary heart disease, macular degeneration, hyposomatotropism, anaemia, fertility disorders, Pubertas praecox, endometriosis, paroxysmal nocturnal haemoglobinuria, as sedatives, for gender reassignment measures, mastodynia, Tourette's syndrome, depression, personality disorders, compulsive disorders, ADHS in children, irritability in foetal alcohol syndrome and autism, delusions, hallucinations, rheumatoid arthritis, Crohn's disease, ulcerative colitis, spondylitis ankylosans (morbus bechterew), psoriasis arthritis, psoriasis, multiple sclerosis, diabetic macular oedema, type 1 diabetes mellitus, and type 2 diabetes mellitus.
